(19) United States
(12) Patent Application Publication
(10) Pub. No.: US 2008/0091108 A1
(43) Pub. Date: Apr. 17, 2008

(54) ULTRASOUND METHODS FOR MIXING LIQUIDS AND COATING MEDICAL DEVICES

(76) Inventor: Eilaz Babaev, Minnetonka, MN (US)

Correspondence Address: Bacoustics, LLC, 5929 BAKER ROAD, SUITE 470, MINNETONKA, MN, 55345, US

(21) Appl. No.: 11/957,557

(22) Filed: Dec. 17, 2007

Related U.S. Application Data
(63) Continuation of application No. 11/204,872, filed on Aug. 16, 2005.

(51) Int. Cl. A61B 8/14

(52) U.S. Cl. 600/462

(57) ABSTRACT

Ultrasound methods for mixing two or more different liquids are disclosed. The ultrasound methods may mix varied components including drugs, polymers, and coatings for application to a variety of medical device surfaces. The disclosed method and apparatus can generate a proper mixture which is uninterruptedly / continuously delivered to the surface of the medical device. The apparatus may include specific ultrasound transducer tip configurations which may allow for the mixing of different liquids in a mixing chamber located inside of the vibrating tip. The apparatus and methods of the present invention may mix different drugs, applying them to a medical device such as a stent surface using the different effects like ultrasound cavitation and radiation forces. Furthermore, the disclosed methods and apparatus may generate a mixture and may deliver a targeted, gentle, highly controllable dispensation of continuous liquid spray which can reduce the loss of expensive pharmaceuticals.

### CROSS REFERENCE TO RELATED APPLICATIONS

This application is a continuation of and claims benefit of U.S. Application Ser. No. 11/204,872, filed Aug. 16, 2005, the teachings of which are incorporated herein by reference.

### BACKGROUND OF THE INVENTION

The present invention relates to the coatings for medical devices and, more particularly, to apparatus and methods using ultrasound energy for mixing two or more different liquids and coating any medical device surfaces. The term “medical device” as used in this application includes stents, catheters, synthetic blood vessels, artificial valves or other similar devices amenable and benefited from spray coating. For clarity, understandability and by way of example, the term “stent” in this application is used interchangeably with the term “medical device”.

A stent is a generally small, cylindrical shaped, mesh tube that is inserted permanently into an artery. A stent helps hold open an artery so that blood can flow through it. Stents can generally be divided into two categories: a) bare metallic stents; and b) drug eluting stents. Drug-eluting stent contain materials that provide therapeutic benefits such as potentially reducing the chance the artery will become blocked again.

Stents are generally tubular in design made up of fine mesh and/or wire having a small diameter and defining a large number of narrow spaces between various components. Frequently, stents are coated with a range of materials utilizing various methodologies and for various reasons. Because of their specific construction, designs and materials, uniformly coating the inner and outer surfaces of the stent, repeatably with no webbing, stringing and with controllable dosage of drug-polymer coating has been problematic.

Examples of patents disclosing stents include U.S. Pat. No. 4,739,762 by Palmaz; U.S. Pat. No. 5,133,732 by Wiktor; U.S. Pat. No. 5,292,331 by Boneau; U.S. Pat. No. 6,908,622 by Barry et al.; U.S. Pat. No. 6,908,624 Hossayniy et al.; and U.S. Pat. No. 6,913,617 by Reiss.

There are a variety of U.S. Published Patent Applications related to stent coatings, including, for example: U.S. Pat. Pub. No. 2003/0225451 A1 by Sundar; U.S. Pat. Pub. No. 2004/0215336 A1 by Udipi, et al.; U.S. Pat. Pub. No. 2004/0224001 A1 by Pacetti, et al.; U.S. Pat. Pub. No. 2004/0234748 A1 by Stenzel; U.S. Pat. Pub. No. 2004/0236399 A1 by Sundar; and U.S. Pat. Pub. No. 2004/0254638 A1 Byun.

According to above-mentioned patents and applications, the coating have been applied to the surface of stents from both inside and outside by different methods, such as mechanical coating, gas spray coating, dipping, polarized coating, electrical charge (electrostatic) coating, ultrasound coating, etc. Some of them like U.S. Pat. No. 6,656,506 utilize a combination of dipping and spraying). Several of them utilize the ultrasound energy, such as, for example, U.S. Pat. No. 6,767,637; and U.S. Pat. Pub. No. 2005/0064088 for ultrasound spraying. In another method, U.S. Pat. No. 5,891,507 discloses coating the surface of a stent by dipping in ultrasonic bath.

Despite these coating technologies and methods, these related technologies have numerous shortcomings and problems. For example, non-uniformity of coating thickness, webbing, stringing, bare spots on the stent surface, drug wasting, over spray, difficulties with control of drug flow volume, adhesivity problems, long drying time and a need for sterilization/disinfection, among others.

Ultrasonic sprayers (U.S. Pat. Nos. 4,153,201, 4,655,393, and 5,516,043) typically operate by passing liquid through the central orifice of the tip of an ultrasound instrument. Known applications include the use of a gas stream to deliver aerosol particles to coating surface. Prior art systems are being used for ultrasonic stent coating by delivering aerosol particles via air jet or gas stream.

Among prior gas ultrasound sprayers are wound treatment applications (U.S. Pat. No. 5,076,266; 6,478,754; 6,569,099; 6,601,581; 6,663,554), which are creating the spray. USSR Patent No. 1237261, issued for Babaev in 1986 can mix the different liquids outside of the ultrasound transducer tip.

Typically, stents need to be coated with a drug and/or polymer in a single layer. Current techniques require the drug or polymer be mixed before coating. This can lead to timing issues such as when a polymer is polymerizes after mixing.

Accordingly, there is a need for a method and device for mixing two or more different drugs with the polymers and defect-free, controllable coating process of the stents.

### SUMMARY OF THE INVENTION

According to the present invention, ultrasonic methods and apparatus for coating medical devices such as stents are described. The term “medical device” as used in this application includes stents, catheters, synthetic blood vessels, artificial valves or other similar devices amenable and benefited from spray coating. For clarity, understandability and by way of example, the term “stent” in this application is used interchangeably with the term “medical device”. The present ultrasonic method and apparatus may provide a proper mixing of two or more different liquids in a mixing chamber defined by an ultrasound transducer tip. The apparatus in accordance with the present invention may create the uniform, gentle and targeted spray for coating of the surface.

In one aspect, the present invention is directed to uninterruptedly mix different liquids and coat medical devices with a controllable thickness layer without webbing and stringing.

In another aspect, the present invention may provide an apparatus including a mixing chamber located inside of the ultrasound transducer tip. A controlled amount of different liquids from different reservoirs may be provided to the mixing chamber of the ultrasonic tip. The ultrasonic tip may be cylindrical, rectangular or otherwise shaped to create the proper mixture. The mixture created may be delivered to the distal end of the tip via a central orifice to create a fine spray.

Liquid may be controllably delivered into the mixing chamber using precise syringe pumps through capillary and/or gravitational action. When using syringe pumps, the amount of liquid delivered may be approximately the same volume or weight as the coating layer.

A method of the present invention for coating medical devices including stents can create a desired mixture inside of an ultrasonic tip from different liquids, drugs, polymers, among other materials and can provide uninterruptedly sprays to the surface.

Methods in accordance with the present invention may also use a number of acoustic effects of low frequency ultrasonic waves, such as cavitation, micro streaming, and standing waves inside of the mixing chamber in the ultrasonic tip, which are not typically utilized in liquid mixing or coating technologies.

The method may include spinning of the stent and moving of the ultrasound mixing and coating head during the coating process to create special ultrasonic-acoustic effects, which will be describe in details below. All coating operations run with special software programs to achieve the high quality results.

The method and apparatus can mix different liquids such as drugs, polymers, etc., and coat rigid, flexible, self expanded stents made by different materials.

A method also may include directing the further gas flow onto the mixing and coating area. The gas flow may be hot or cold and directed through the mixing chamber and/or spray within particles or separately.

The disclosure includes various embodiments of ultrasonic tips, which allow mixing of different liquids to uninterruptedly create the spray.

The ultrasound frequency may be between 20 KHz and 20 MHz or more. Preferable frequency is 20 KHz to 200 KHz, recommended frequency is 30 KHz. The rate of ultrasound waves amplitude may be between 2 micron and 300 micron or more. Thereby, there is provided a method and device for uninterruptedly ultrasound stent coating with proper mixing of different liquids with no webbing and stringing.

One aspect of this invention may be to provide a method and device for mixing two or more different liquids.

Another aspect of the invention may be to provide a method and device for mixing two or more unmixable liquids.

Another aspect of the invention may be to provide an improved method and device for mixing two or more different drugs, polymers, or drug with the polymer for coating of medical implants such as a stents.

Another aspect of this invention may be to provide a method and device for mixing two or more different liquids, such a drugs, polymers or a combination of drugs with the polymer and coating of stents using ultrasound.

Another aspect of this invention may be to provide a method and device for mixing two or more different drugs with the polymers, that provides controllable thickness of coating layer.

Another aspect of the invention may be to provide a method and device for simultaneous mixing of different liquids, creation of continuous, uniformed, directed spray for the proper mixture coating of stents.

Another aspect of the invention may be to provide a method and device for simultaneous mixing of different liquids, creation of continuous, uniformed, directed spray from proper mixture coating of stents, that avoids the coating defects like webbing, stringing, etc.

Another aspect of the invention may be to provide a method and device for simultaneous mixing of different liquids, creation of continuous, uniformed, directed spray for the proper mixture coating of stents, which increases the adhesivity property of stents without the use of chemicals. Another aspect of the invention may be to provide method and device for simultaneous mixing of different liquids, creation of continuous, uniformed, directed spray from proper mixture coating of stents that provides drying of the coating layer along the longitudinal axis of the structure simultaneously with the coating process.

Another aspect of the invention may be to provide a method and device for simultaneous mixing of different liquids, creation of continuous, uniform, directed spray from a proper mixture of a stent coating, that provides sterilization of the coating layer along the longitudinal axis of the structure simultaneously with the coating process.

Another aspect of invention may be to provide a method and device for creation of uninterrupted process of proper mixing two or more different liquids, creating the spray and coating the surface.

These and other aspects of the invention will become more apparent from the written description and figures below.

### BRIEF DESCRIPTION OF THE DRAWINGS

The present invention will be shown and described with reference to the drawings.

FIG. 1 is a cross sectional view of an embodiment of an ultrasonic mixing apparatus in use with the spray according to the present invention.

FIG. 2 illustrates a cross section of an embodiment of the apparatus with the delivery of liquid directly to the mixing chamber inside of ultrasonic tip according to the present invention. Liquid delivery tubes are located on one platan, perpendicular to the longitudinal axis of the tip.

FIG. 3 illustrates the cross section of an embodiment of an apparatus with the delivery of liquid directly to the mixing chamber inside of the ultrasonic tip according to the present invention. Liquid delivery tubes are located on the platan, along ultrasonic tip's longitudinal axis.

FIG. 4 is an illustration of a cross section of an embodiment of an apparatus with the threaded mixing chamber inside of the ultrasonic tip.

FIG. 5 illustrates a cross section of an embodiment of the apparatus with the delivery of one liquid to mixing chamber through a central orifice of ultrasound transducer, and another liquid through the tube, perpendicular to ultrasonic tip's axis.

FIGS. 6A and 6B illustrate embodiments of the mixing chambers in expanded cross section having rounded radiation walls.

FIG. 7 illustrates embodiments of ultrasonic tips which are A) expanded flat, B) conical shape, C) exponential D) outside rounded, E) inside rounded-focused, and F) rectangular distal end configurations.

### DETAILED DESCRIPTION OF THE INVENTION

The present invention provides a method for the coating of medical devices. Under the preferred embodiment, an apparatus including an ultrasonic tip 1 defining a mixing chamber 4 is used. Preferred embodiments of the present invention in the context of a method and apparatus are illustrated throughout the figures. Those skilled in the art will immediately understand the advantages for mixing of two or more different liquids such as a drugs and/or polymers and uninterruptedly coating the stent that will be provided by the present inventions upon review of the disclosure.

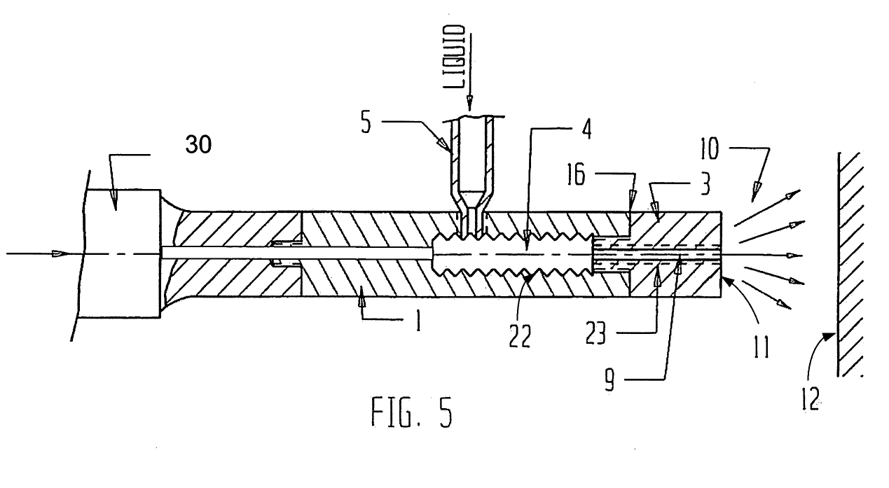

The ultrasonic tip 1 uses ultrasonic energy provided by an ultrasound transducer 30 to mix materials and coat medical apparatus. The methods are particularly useful when applied to coating stents and other devices having intricate details and complex shapes. Ultrasonic tips 1 in accordance with the present invention can provide highly controllable precise mixing of two or more drugs and polymers. The fine, targeted spray allows the coating of stents without substantial webbing, stringing and wasting the expensive drug through improved mixing. The present invention provides a novel ultrasonic tip 1 and method for mixing two or more different fluid to coat a stent. Embodiments of ultrasonic tip 1 in accordance with the present invention are illustrated in FIGS. 1 to 7. According to present invention, ultrasonic tip 1 includes a mixing chamber 4 inside of the ultrasonic tip 1.

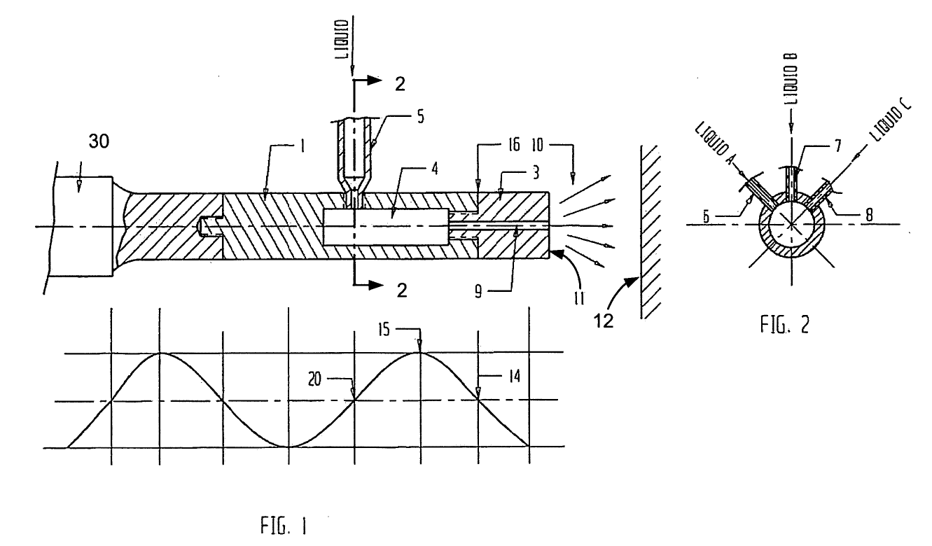

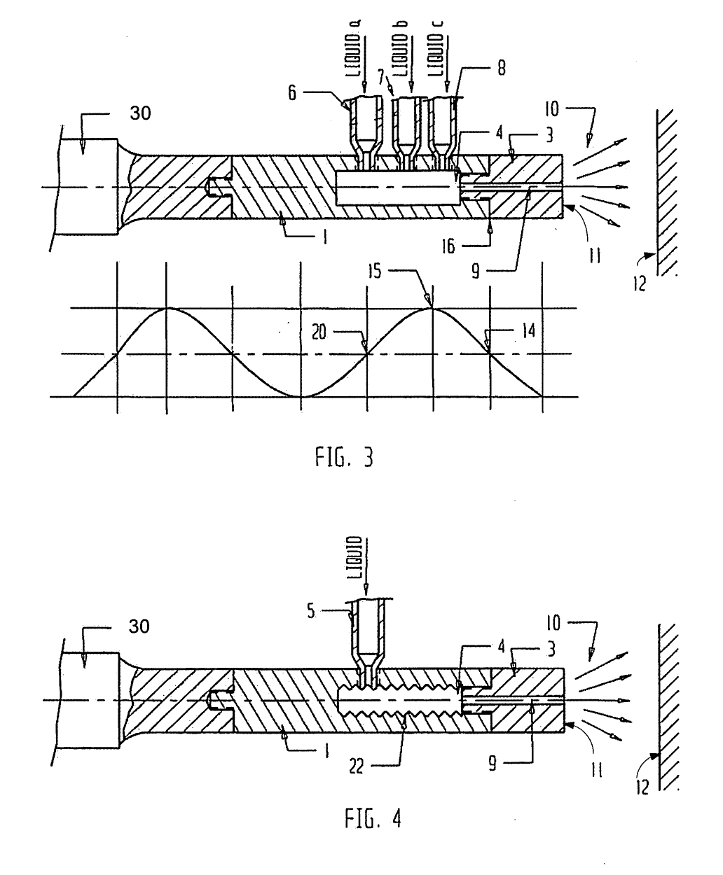

The mixing chamber 4 provides ultrasonically active space for mixing of different liquids under acoustic forces including cavitation phenomena which can occur inside of chamber 4. Typically, chamber 4 is of a cylindrical shape about the longitudinal axis of the ultrasonic tip 1. Typically, this cavitation phenomena occurs between walls 18 and 19 of the chamber perpendicular to the longitudinal axis. One or more syringe pumps (not shown) may be provided for delivery of different liquids into chamber 4 through tubes 5, 6, 7, 8, (FIG. 2), located on the platan perpendicular to longitudinal axis. Liquid delivery tubes 6, 7, 8 may be located along longitudinal axis (FIG. 3) is one of the embodiment of present invention. Mixtures of drugs and/or polymers may be delivered in an uninterruptedly fashion to a radiation surface 11 of tip 1 through an orifice 9 for creation of spray 10 and delivery to coating surface/stent 12. Diameter of orifice 9 preferably must be less than the diameter of the mixing chamber. To simplify manufacture, an ultrasound transducer 30 an ultrasonic transducer tip 1 may include a distal end part 3 which is attached via threads on planar 16 to form the chamber 4. Distal end part 3 may be provided with a different diameter of central orifice 9 to create the spray 10 in the needed particle size. To avoid the loose of distal end part 3 has to be attached to tip 1 preferably on amplitudes node point 14. Liquid delivery tubes (5, 6, 7, 8) also have to be attached to tip 1 preferably on amplitudes node point 20. To achieve a high quality mixture, the mixing chambers center can match or be near to the amplitudes anti node point 15.

It is important to note that a gas stream with a different temperature can be delivered into mixing chamber 4 through one of the tubes (5, 6, 7, 8) to improve liquid mixing and spray coating process. This can change the spray volume, spray quality and may expedite the drying process.

When different liquids (a, b, c) are provided into mixing chamber 4, distal wall 19 under ultrasound radiation force delivers liquid flow forward. Retrograded or ricocheted liquid from proximal wall 19 encounters incoming liquid flow and creates the proper mixture from the ultrasound radiation/pressure forces and cavitation.

After the mixing chamber fills with the fluid, the ultrasound pressure forces the mixture through central orifice 9 to create spray 10 which is delivered to radiation surface 11. As the liquids are delivered and the tip vibrates, the mixing and spray coating process are occurring uninterruptedly.

In one aspect of the present invention, for more effective and proper mixing process, mixing chamber 4 consists of at least one thread 22, groove ring or a waved shape (See FIG. 5). In this case the tooth of the tread acts as a mixing blade or spoon, forcing the different liquids to be mixed with the ultrasonic energy. The distal end orifice 9 also can be threaded 23 to provide a better mixing process.

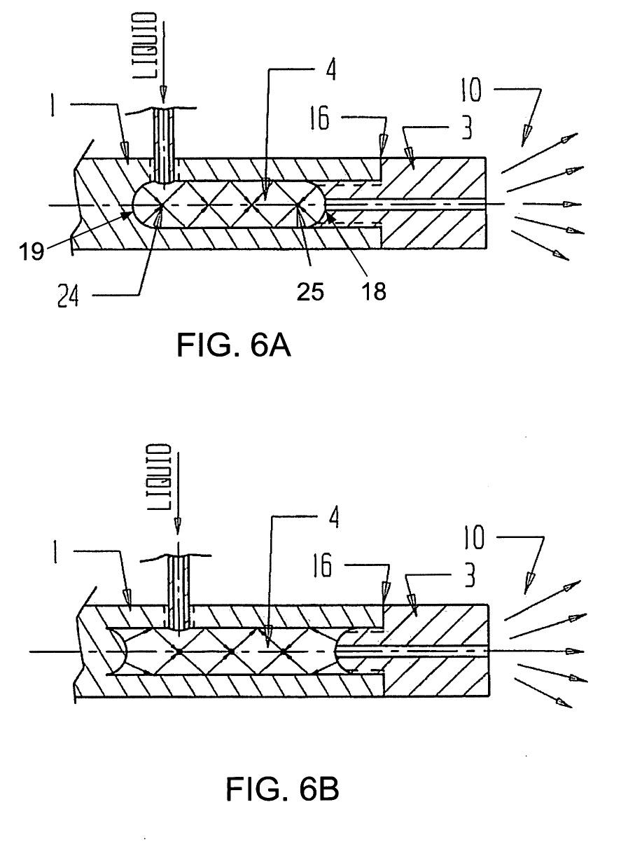

In another aspect (See FIG. 6), for a more effective and proper mixing process, mixing chambers distal wall 18 and proximal wall 19 can be rounded inside (See FIG. 6A) to create the focused ultrasonic effect, which is much more powerful than the unfocused alternative. FIG. 6A shows the proximal focus point 24 and the distal focus point 25. The chamber walls also can be convex (See FIG. 6B) for the creation of a powerful cavitation effect to achieve an improved mixing process. In this case ultrasound waves being reflected from radial cylindrical walls of the mixing chamber force the different liquid particles toward each other. This provides improved mixing of different liquids under ultrasound cavitation and radiation pressure.

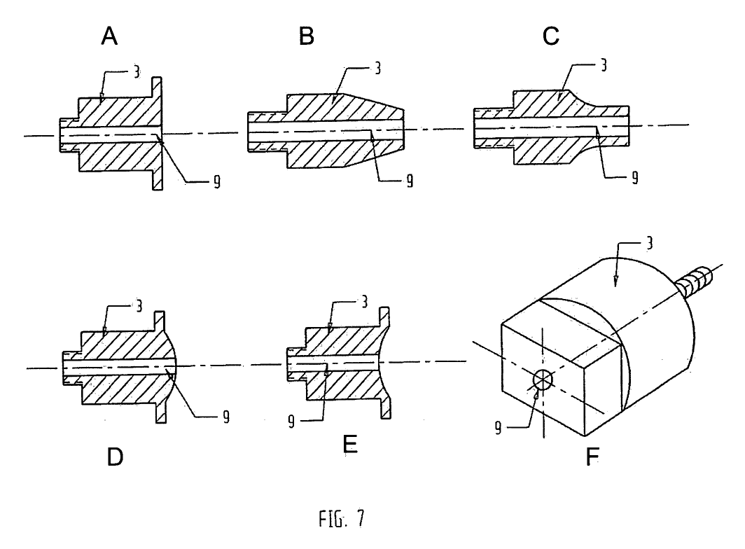

FIGS. 7A-E provide an illustration of several ultrasonic tip embodiments A) expanded flat, B) conical shape, C) exponential, D) outside rounded, E) inside rounded-focused and F) rectangular distal end configurations. These configurations allow control of the spray angle and coating quality depending on the coating requirements.

We claim:

1. A method for coating a medial device comprising the steps of:
emitting ultrasonic energy using a device having:
an ultrasound transducer having an ultrasonic tip;
the ultrasonic tip having a central axis and a distal end;
the ultrasonic tip also having a mixing chamber inside the ultrasonic tip;
the mixing chamber having a proximal wall, a distal wall and at least one radial wall;
at least one tube positioned along a perpendicular plane to the central axis;
the tube to deliver a first fluid and a second fluid to the mixing chamber;
the mixing chamber generating a mixture; and
the ultrasonic tip having a central orifice at the distal end for discharging the mixture;
delivering the first and second fluid into the mixing chamber;
generating a mixture from the fluids within the mixing chamber;
spraying the mixture through the central orifice onto the medical device; and
coating the medical device.

2. The method of claim 1 wherein the step of generating the mixture within the mixing chamber improves the adhesivity of the mixture.

3. The method of claim 1 in which at least one of the group consisting of the first fluid or the second fluid is a gas.

4. The method of claim 1 wherein the first fluid is a liquid and the second fluid is a gas.

5. The method of claim 1 wherein the step of generating a mixture uses ultrasound cavitation to enhance mixing.

6. The method of claim 1 wherein the step of generating a mixture uses ultrasound focusing to enhance mixing.

7. The method of claim 1 wherein the step of generating a mixture uses ultrasound standing waves to enhance mixing.

8. The method of claim 1 in which ultrasound microstreaming is used to enhance mixing.

9. The method of claim 1 in which the side wall of the device emitting ultrasound energy has at least one thread.

10. The method of claim 1 in which the central orifice of the device emitting ultrasound energy has at least one thread.

11. The method of claim 1 in which the tube is attached to the ultrasound tip of the device emitting ultrasound energy approximately on a node point.

12. The method of claim 1 in which the distal end tip has a geometric confirmation selected from the group consisting of convex, concave tapered or flat.

13. The method of claim 1 in which the proximal wall of the mixing chamber is flat.

14. The method of claim 1 in which the distal wall of the mixing chamber is flat.

15. The method of claim 1 in which the proximal wall of the mixing chamber is convex.

16. The method of claim 1 in which the distal wall of the mixing chamber is convex.

17. The method of claim 1 in which the proximal wall of the mixing chamber is concave.

18. The method of claim 1 in which the distal wall of the mixing chamber is concave.

19. The method of claim 1 in which delivering the first fluid to the mixing chamber occurs axially at the proximal wall of the mixing chamber.

20. The method of claim 1 having the additional step of heating at least one of the group of the first fluid or the second fluid.

21. The method of claim 1 wherein the ultrasound transducer vibrates the ultrasonic tip at a frequency in the range of 20 KHz to 20 MHz.

22. The method of claim 1 wherein the ultrasound transducer vibrates the ultrasonic tip at a frequency of approximately 30 KHz.

23. The method of claim 1 wherein the ultrasound transducer vibrates the ultrasonic tip at an amplitude within the range of 2 microns to 300 microns.
